(19) United States
(12) Patent Application Publication
(10) Pub. No.: US 2005/0032042 A1
(43) Pub. Date: Feb. 10, 2005

(54) Anti-retroviral analysis by mass spectrometry

(76) Inventor: Steven J. Soldin, Washington, MD (US)

Correspondence Address: TORYS LLP, 79 WELLINGTON ST. WEST, SUITE 3000, TORONTO, ON, M5K 1N2, CA

(21) Appl. No.: 10/814,244

(22) Filed: Apr. 1, 2004

Related U.S. Application Data
(60) Provisional application No. 60/462,672, filed on Apr. 14, 2003.

(51) Int. Cl. C12Q 1/70; G01N 33/00

(52) U.S. Cl. 435/5; 436/91

(57) ABSTRACT

Methods for the simultaneous or sequential analysis and quantification of a plurality of antiretroviral analytes in a complex biological matrix by mass spectrometry are disclosed. The methods require minimal sample size, minimal preparation time and allow for rapid through-put. The system is particularly useful in therapeutic drug monitoring.

This application claims the benefit of the filing date of U.S. Provisional application Ser. No. 60/462,672 filed Apr. 14, 2003 under 35 U.S.C. 119(e).

### FIELD OF THE INVENTION

The present invention combines the fields of clinical medicine and analytical chemistry. In particular the invention relates to the analysis of multiple analytes within a complex biological matrix by mass spectrometry, specifically where the analytes are antiretroviral drugs, including protease inhibitors (PIs), nucleoside reverse transcriptase inhibitors (NRTIs), nucleotide reverse transcriptase inhibitors (NtRTIs), non-nucleoside reverse transcriptase inhibitors (NNRTIs) and fusion inhibitors (FIs).

### BACKGROUND OF THE INVENTION

Quantification of the therapeutic level of drugs in the body is an important element of therapeutic drug monitoring (TDM), a process with numerous applications in clinical medicine. TDM has particular relevance in the treatment of Human Immunodeficiency Virus (HIV), which has become increasingly sophisticated and complex [1-3].

Over the past several years, there has been a rapid increase in the number of marketed anti-HIV drugs. Table 1 lists some of the currently marketed antiretrovirals in their classes, with several more expected to reach the market in the near future. Cocktails of 3 to 4 drugs are used routinely in anti-HIV therapy. The specific choice of drugs depends on a number of factors, such as drug resistance, tolerability, drug interactions and effectiveness of treatment [4-6]. Combination of these factors (especially drug resistance) often leads to the need to alter the drugs prescribed to each patient. In such situations, therapeutic monitoring becomes an important factor.

The pharmacokinetics of many of the anti-HIV drugs are complex and sometimes unpredictable. Most of these drugs largely undergo oxidative CYP-3A4-mediated metabolism, which occurs primarily in the liver and gastrointestinal tract and is prone to numerous drug interactions and high inter and intra-individual variability.

Although some of the anti-HIV drugs do not require TDM themselves, complicated regimens and drug cocktails with multiple drug interactions may justify TDM [7,8]. In addition, TDM may be the only way to effectively verify compliance, an issue which has been shown to be critical in HIV therapy [9]. The literature has shown that TDM of antiretrovirals is very useful for the PIs and for the NNRTIs while its use for the NRTIs may be predominately to assess patient compliance [10,11,12]. To date, no assay reported in the literature has covered the full spectrum of antiretrovirals. In addition, a large difference in trough concentrations of different HIV drugs represent a further challenge to development of a universal assay.

Traditionally, in clinical laboratories, TDM was performed by the use of immunoassay techniques and high performance liquid chromatography (HPLC).

However, immunoassay techniques are disadvantageous for the following reasons:
(1) Immunoassays are specific to each analyte. Therefore every drug must be analyzed separately.
(2) Numerous immunoassay kits must be purchased and procedures must be learned for each drug being analyzed by an immunoassay.
(3) Various instruments, such as gamma counters and photon counters, must be purchased to read the results from the immunoassays.
(4) The kits for immunoassays can be expensive.
(5) For some analytes, current immunoassays show, approximately, a 15 fold difference in results using kits from different manufacturers [13].
(6) In the case of a radioimmunoassay, precautions are necessary because of the radioisotopes involved.
(7) Immunoassays lack specificity and have problems with cross-reactivity, which tends to overestimate the concentration of the parent drug [13].

Recently, a method has been developed for the simultaneous determination of eight antiretroviral drugs (five PIs and three NNRTIs) by HPLC ion trap mass spectrometry [15]. The authors of this method obtained calibration curves with a range of between nearly 20 and 10,000 ng ml⁻¹, and intra and interday precision of less than 15%. They also declared that their assay allows for elution and drug analysis within 15 minutes using 250,μL of human plasma.

However, HPLC methods are disadvantageous for the following reasons:
(1) The method requires laborious and time consuming liquid-liquid extraction.
(2) The method lacks sensitivity and requires large serum volumes.
(3) Present HPLC methods are not applicable to NRTIs.
(4) Many different HPLC methods must be employed.

Researchers have applied liquid chromatography mass spectrometry methods to evaluate the plasma levels of various drugs and a number of assays for simultaneous quantification of various groups of anti-HIV drugs (especially PIs) have been reported [16-25]. However, none of these methods can assay PIs, NRTIs, NtRTIs, NNRTIs and FIs simultaneously.

Similarly, other liquid chromatography tandem mass spectrometry methods have been developed to measure the levels of up to five PIs in human plasma [23-24 ]. One of these methods involved precipitating plasma proteins and injecting the supernatant into a liquid chromatography column. The authors demonstrated a dynamic range of between 5.0 and 10,000 ng ml⁻¹, but used two calibration curves for each assay and 100,μL of plasma was required.

### SUMMARY OF THE INVENTION

The invention provides a fast, simple and accurate method for simultaneously or sequentially analyzing at least two antiretroviral drugs, including PIs, NRTIs, NtRTIs, NNRTIs and FIs, comprising ionizing the drugs and analyzing the drugs by mass spectrometry. Antiretroviral drugs from a variety of classes as shown in Table 1, can be simultaneously or sequentially analyzed by this method.

Accordingly, the invention provides a method for mass spectrometric analysis of a sample comprising at least two antiretroviral drugs from at least two classes of antiretroviral drugs, the method comprising the steps of (a) providing a sample of at least two antiretroviral drugs from at least two classes of antiretroviral drugs, (b) introducing the sample comprising at least two antiretroviral drugs from at least two classes of antiretroviral drugs into a mass spectrometer, and (c) analyzing the sample using the mass spectrometer. The mass spectrometer can be a tandem-mass spectrometer, selected from the group consisting of API 2000™, API 3000™ and API 4000™.

The method can further comprise a step of deproteinating the sample. This can be done by adding acetonitrile to the sample, vortexing the sample and centrifuging the sample. Alternatively, this can be done by subjecting the sample to precipitation with an agent selected from the group consisting of methanol, ethanol and salt.

The classes of antiretroviral drugs can be selected from the group consisting of PIs, NRTIs, NtRTIs, NNRTIs and FIs. The antiretroviral drugs can be selected from the group consisting of amprenavir, indinavir, nelfinavir, ritonavir, saquinavir, lopinavir, abacavir, didanosine, lamivudine, stavudine, zalcitabine, zidovudine, delavirdine, efavirenz, nevirapine, tanofavir, atazanavir, peptide T and T-20.

The drugs can be analyzed in a variety of matrices, including plasma, serum, saliva and urine. For plasma and serum, both free and total drug analysis can be performed. Free form analysis can be performed for saliva, and for urine, the excreted amount can be measured.

The method can further comprise a step of cleaning the sample, for example by introducing the sample to a chromatography apparatus and eluting the sample.

The invention also provides a method for therapeutic drug monitoring in patients with HIV infection, comprising providing a sample comprising at least two antiretroviral drugs from at least two classes of antiretroviral drugs, (b) introducing the sample comprising at least two antiretroviral drugs from at least two classes of antiretroviral drugs into a mass spectrometer, and (c) analyzing the sample using the mass spectrometer.

The invention also provides a system for the mass spectrometric analysis of a sample comprising at least two antiretroviral drugs from at least two classes of antiretroviral drugs, comprising (a) reagents for deproteinating the sample, (b) reagents for analyzing the sample by mass spectrometry, and (c) a mass spectrometer.

The invention also provides a kit, comprising the various reagents required for simultaneously or sequentially analyzing, within a sample, at least two antiretroviral drugs, including PIs, NRTIs, NtRTIs, NNRTIs and FIs. The kit may include a reagents for deproteinating the sample, reagents for analyzing the sample by mass spectrometry, instructions for analyzing the sample using a mass spectrometer. The kit may further include mobile phase solutions, a chromatography column and a quality control specimen, standard solution of the drugs of interest, compounds as internal standards, HPLC column. For example, the kit can be used to test a sample for one or more of the commonly administered drugs listed in Table 1.

The invention provides a use of a mass spectrometer for sequentially or simultaneously analyzing a sample containing at least two antiretroviral drugs from at least two classes of antiretroviral drugs. The mass spectrometer may be a tandem mass spectrometer. The sample may be any sample and includes but is not limited to, serum, plasma, urine or saliva.

Various ionization techniques are used in combination with analysis by mass spectrometry to arrive at this invention. Chromatographic separation is not required, although chromatography may be employed to clean the sample of impurities, such as salts.

The invention provides a system for the fast, simple and accurate analysis of a plurality of antiretroviral drugs comprising: reagents for the preparation of the sample and reagents to perform the analysis on a mass spectrometer, and the mass spectrometer to perform the analysis.

Accordingly, there is provided a method that allows for the simultaneous or sequential analysis of a plurality of antiretroviral drugs in a sample, comprising: obtaining a sample, deproteinating the sample, cleaning the sample, and analyzing and quantifying analytes within the sample by mass spectrometry.

Accordingly, there is also provided a method for the simultaneous or sequential analysis of a plurality of antiretroviral drugs, including PIs, NRTIs, NtRTIs, NNRTIs and FIs in a sample, including a sample of plasma, serum, saliva or urine, comprising: obtaining a sample, deproteinating the sample, cleaning the sample, and quantifying analytes within the sample by mass spectrometry.

Accordingly, there is also provided a method for the simultaneous or sequential analysis of a plurality of antiretroviral drugs, including PIs, NRTIs, NtRTIs, NNRTIs and FIs in a sample, including a sample of plasma, serum, saliva and urine, comprising: obtaining a sample, deproteinating the sample, cleaning the sample, and quantifying analytes within the sample by a mass spectrometry technique, absent chromatographic separation of the analytes.

There are several advantages to this invention:
(1) It permits the simultaneous or sequential quantification of a plurality of antiretroviral drugs, including PIs, NRTIs, NtRTIs, NNRTIs and FIs. Quantification of a range of antiretrovirals was beyond the reach of previous chromatography mass spectrometry methods, because of the high sensitivity and specificity demanded by the large variation of the various antiretroviral drug classes (See Table 2).
(2) The invention requires minimal sample preparation time. For example, after deactivating the HIV virus by a method known to one skilled in the art, preparing a sample of plasma for analysis can be completed within 20 minutes.
(3) The procedure does not require a large sample size. For example, a plasma sample as small as 80 μL permits quantitation of a plurality of HIV/AIDS drugs and allows for drug quantitation in infants and neonates.
(4) The procedure uses simple sample preparation techniques that are easy to use and highly reproducible, with good inter and intraday precision, below at least 7% for all analytes.
(5) The invention permits the analysis of antiretrovirals in a sample of saliva or urine which permits simple sample acquisition and the remote submission of samples to a clinic for analysis. In other clinical methods, samples are taken by invasive means directly from the patient in a clinic [16].
(6) The time to complete an analysis of an batch of samples, from initiation to completion is about 15 minutes, which is far less time than alternative methods. Alternative methods take many hours.
(7) The invention is highly accurate over a wide range of concentrations, with calibration curves that are linear from at least 2 to 10,000 ng ml⁻¹ for stavudine, didanosine, zalcitabine and AZT; 100 to 10,000 ng ml⁻¹ for tenofovir; and from 10 to 10,000 ng ml⁻¹ for all other antiretroviral drugs.

### DETAILED DESCRIPTION OF THE EXEMPLIFIED EMBODIMENTS

The invention provides methods for the simultaneous or sequential analysis of a plurality of antiretroviral drugs, including PIs, NRTIs, NtRTIs, NNRTIs and FIs, as well as variations, analogues and improvements thereto. A list of sample antiretrovirals that may be so analysed is provided in Table 1.

Sample Preparation

Any biological sample can be used. For example plasma, serum, urine or saliva can be used. A sample size of about 80 μL can be used.

When required, the sample is de-proteinated, according to conventional techniques known to those skilled in the art. For example, a sample can be de-proteinated with acetonitrile, containing an internal standard as selected by one skilled in the art, followed by vortexing and centrifugation. Other methods of de-proteinization include precipitation with methanol, ethanol or salts.

Chromatography of Sample

The supernatant is introduced to a chromatography apparatus and eluted. In the case of a liquid chromatography apparatus, the column may be a C-18 column. No chromatographic separation is necessary for quantification where a tandem mass analyzer is employed. Where a tandem mass analyzer is employed, the matrix can be cleaned of impurities by a chromatography column, or by other methods known to those skilled in the art. All the compounds of the sample may adhere to the column. The column is then washed and the compounds are eluted out. Optionally, a built in switching valve may be used. The system is easy, provides rapid analysis and is fully automated. Optionally, a C18 Sep-Pack ™ may be used to clean the sample.

Mass Spectrometry of Sample

The sample is then introduced into a mass spectrometer.

The following mass spectrometers may be used: any tandem-mass spectrometer, including the API 2000™, the API 3000™ and the API 4000™.

Instrumentation and Ionization Techniques

The drugs are subjected to ionization. Various ionization techniques can be used. Preferably, atmospheric pressure chemical ionization is utilized, preferably using a heated nebulizer.

Ionization may be performed by utilizing the mass spectrometer in the negative or the positive mode, depending on a particular analyte's tendency to give rise to a particular ion form, as is known to those skilled in the art. Optimally, the choice of negative or positive mode is made in accordance with Table 3. Optionally, cimetidine may be chosen as an internal standard, as it is suitable for both ionization modes, further simplifying the procedure.

As was discussed elsewhere [25], the use of the APCI source minimizes the potential for ionization suppression and matrix effects. Co-elution of the analytes and the internal standard further helps to overcome these problems, and eliminates the need to use internal standards structurally related to the analytes, which would be impossible considering the number of monitored drugs. Optionally, cimetidine may be chosen as an internal standard, as it is suitable for both ionization modes, further simplifying the procedure.

If the combination of drugs taken by the AIDS patient is not known, monitoring a plurality of antiretrovirals drugs (for example, monitoring seventeen or more) may be needed. This would result in a simultaneous monitoring of at least fourteen ion transitions in the positive mode. In such a case care should be taken to adjust dwell times and delays between ion transitions so that there are at least ten to twelve data points for each monitored peak. Failure to meet this requirement may result in inadequate peak integration and considerably worse precision. This, however, can occur only in the most complicated scenario, when the analyst does not know which anti-HIV drugs were taken by the patient, which is rarely the case in clinical practice.

Analysis

Analytes, such as the antiretrovirals analysed by the present method, are identified in a mass spectrometer as is known to those skilled in the art. For example, analytes may be analyzed on the basis of the mass to charge ratio of their fragment ions. Preferably, an internal standard may be used, as is known to those skilled in the art. Calibration curves for known concentrations of drugs are established for comparison.

### EXAMPLES

The invention may be demonstrated using the following four examples, provided to demonstrate but not limit the embodiments of the present invention. In the first example, 15 drugs are analyzed with the aid of a methanol standard. In the second example, 16 drugs (comprising the 15 analyzed in Example 1, and tenofovir, a NtRTI) are analyzed, with the aid of a serum standard. The third example illustrates how the invention might be applied to encompass new antiretroviral compounds, such as the FI T-20. The fourth example illustrates correlations between free plasma drug concentration and saliva drug concentration with total plasma drug concentration for a number of PIs, NRTIs and NNRTIs.

### Example 1

### Methanol Standard Matrix

Standards and Chemicals

Standards of zalcitabine (ddC), didanosine (ddI) and zidovudine (AZT) were purchased from Sigma (St. Louis, Mo., USA). Primary standards of efavirenz, indinavir, nelfinavir, nevirapine, ritonavir, saquinavir, lamivudine, abacavir and stavudine were obtained from the National Institutes of Heath (NIH) Aids Reagent Reference Program (McKessonHBOC BioServices, Rockville, Md.) while standards for amprenavir, delavirdine and lopinavir were isolated from commercially available tablets/capsules and characterized by H-NMR, UV spectroscopy and elemental analysis. Methanol, acetonitrile, and ammonium acetate were purchased from Sigma and were of HPLC grade.

Standard Solutions and Calibration Curves

Stock solutions were prepared separately to obtain concentrations of 0.1 mg mL⁻¹ for each drug (total of 15 drugs). Methanol was used as a solvent. Working Standard Solution was prepared by mixing equal amounts of stock solutions of each drug and diluting 1:4 with methanol to obtain a solution containing 1.67 μg mL⁻¹ of each drug. A seven-point calibration curve (blank and six calibrators) was prepared for calibration. Along with 50 μL of the internal standard solution, 40 μL of working standard solutions were placed into 1.5 mL Eppendorf conical plastic test tubes, after which 40 μL of blank plasma were added to each test tube. The calibrators were further treated as described under Sample Preparation.

A solution of 0.15 mg L⁻¹ of cimetidine in methanol was used as an internal standard (for both negative and positive MS/MS modes).

Sample Preparation

For sample preparation, 80 μL of serum or heparinized plasma were placed into a 1.5 mL conical plastic Eppendorf test tubes containing 50 μL of internal standard solution and vortexed briefly. Then 200 μL of acetonitrile were added, the test tubes were capped, vortexed vigorously for 30 seconds and cenrtifuged at 14,000 g for 10 minutes. The supernatant was transferred into autosampler vials for injection into the liquid chromatography tandem mass spectrometry system (LC/MS/MS). A doubleblank sample (a sample that contains neither of the standards nor the internal standard) was also prepared with each calibration curve. Sample preparation was performed at room temperature. Plasma obtained from HIV-infected patients was heated for 30 minutes at 56° C. to deactivate the HIV virus [26].

This demonstrates a simple and expeditious method of preparing for analysis a complex matrix containing a plurality of antiretroviral drugs.

LC/MS/MS Setup and Procedure

An API-2000™ tandem mass spectrometer (SCIEX, Toronto, Canada) equipped with atmospheric pressure chemical ionization (APCI, heated nebulizer) source, two Perkin-Elmer PE-200™ series micropumps and autosampler (Perkin-Elmer, Norwalk, Conn., USA) was used to perform the analysis. Data processing was performed on Analyst 1.1 software package (SCIEX). The main working parameters of the mass spectrometer are summarized in Table 4.

The procedure was based on an online extraction/cleaning of the injected sample with subsequent introduction into the mass spectrometer by using a built-in switching valve. 30 μL of the sample were injected onto a Supelco LC-18-DB™ (3.3 mm×3.0 mm, 3.0 μm ID) chromatographic column equipped with Supelco Discovery C-18™ (3.0 mm) guard column (Supelco, St. Louis, Mo., USA). The sample underwent cleaning with an aqueous solution of ammonium acetate (15 mM) at a rate of 1 mL min⁻¹. After 2.4 min of cleaning the switching valve was activated, the column was flushed with methanol at a rate of 1 mL min⁻¹ and the sample was introduced into the mass spectrometer.

Analytes were then quantified by multiple reaction monitoring (MRM) (see Table 5 for MRM transitions). MRM allows for enhanced selectivity through the measurement of parent and daughter ions simultaneously for each of the compounds of interest.

Due to the high selectivity of the tandem mass analyzer, no chromatographic separation was necessary for quantification of the analytes and the analysis was complete less than a minute after activation of the switching valve. Total analysis time was 4.5 min, including equilibration time before the next injection. The procedure was completely automatic and controlled by the Analyst 1.1 software.

Drug Interference Studies

The following commonly used drugs were tested at both their therapeutic and toxic concentrations for potential interference in the procedure described: acetaminophen, amikacin, caffeine, carbamazepine, digoxin, disopyramide, ethosuximide, flecainide, gentamicin, lidocaine, lithium, methotrexate, N-acetylprocainamide, phenobarbital, phenytoin, primidone, procainamide, quinidine, salicylate, theophylline, tobramycin, valproic acid, and vancomycin.

Drug Comparison Studies

Comparison studies were performed for the PIs and NNRTIs using the tandem mass spectrometry method utilized at the BC Center for Excellence in HIV/AIDS.

Quality Control and Proficiency Testing

Serum or plasma spiked with known concentrations of the drugs and at three levels (low, medium and high) were used as daily quality controls. External proficiency testing is available from “International Quality Control Program for Therapeutic Drug Monitoring in HIV Infection” (University Medical Center Nijmegan, Department of Clinical Pharmacy, PO Box 9101, 6500 HB Nijmegan, The Netherlands).

Accuracy and precision were evaluated by analyzing quality control samples at low, medium and high concentrations on 20 different days. Within-run precision (% CV) was below 7% for all analytes. Between-day precision (% CV) was below 10% for all analytes at the tested concentrations. Accuracy (% of weighed-in target concentration measured) ranged between 95% and 105%. The results are summarized in Table 5. The assay was linear over the range of 2 to 2000 ng mL⁻¹ for stavudine, didanosine, zalcitabine and AZT, and 10 to 10000 ng mL⁻¹ for all other drugs. None of the drugs listed above interfered in the method described.

The performance of the method was also compared with other analytical methods used for quantitation of nine of the tested drugs. A total of approximately 600 clinical samples containing various combinations of amprenavir, indinavir, saquinavir, delavirdine, efavirenz, lopinavir, ritonavir nelfinavir, and nevirapine were analyzed by the new method, as well as by a different LC/MS/MS method used routinely for the TDM of HIV drugs in British Columbia. The study was performed in a blinded manner and the laboratories did not share the results until after the completion of the study. Specimens were transported on dry ice and sent by overnight courier. They were stored at −70° C. until analyzed. These drugs are known to be stable at 4° C. for 1 week. The results are summarized in the Table 6. As can be seen from the Table 6, the results correlated very well for all of the tested drugs. Differences in the regression slopes can be explained by the differences in standardization between the two laboratories. Due to the difficulty in obtaining standards from the drug companies [16], both of the laboratories purified the drugs initially from tablets. Gold standards for these drugs were only recently obtained by our laboratory from the NIH and were used to recalibrate our standards, perhaps accounting for some of the differences between the laboratories.

This demonstrates a simple, expeditious and automated method of simultaneously analyzing a plurality of antiretroviral drugs by mass spectrometry. In particular, this demonstrates that the invention is easy and reliable and allows for the simultaneous measurement of any combination of AIDS drugs in less than 5 minutes. This facilitates TDM for both the PIs and NNRTIs.

### Example 2

### Serum Standard Matrix

This example demonstrates how the methods of the present invention can be used for therapeutic drug monitoring (TDM) in the treatment of patients with HIV/AIDS. The analytical component of TDM requires a drug assay with high specificity, small sample volume requirements, reasonable cost and rapid turnaround time. The major reasons for TDM include improving therapeutic efficacy, preventing drug toxicity and verifying patient compliance. Protein binding is an important factor influencing this concentration-response relationship, particularly for the protease inhibitors (27, 34) because changes in the overall binding of these drugs could affect the interpretation of total drug concentrations (27). Analytical criteria for TDM are that a drug assay be available with high specificity, sensitivity, which requires only small sample volumes, at low cost and providing rapid turnaround time (30).

This example modifies the procedure of example 1, for the concurrent measurement of fifteen antiretrovirals by tandem mass spectrometry. The upper limit of the calibration curves were extended to 10,000 ng/mL, and the matrix for standards was changed from methanol to serum. Also, an additional drug tenofovir, a nucleotide reverse transcriptase inhibitor, was included in this example. Calibration curves for all 16 anti-HIV drugs showed good linearity between a concentration range of 100-10,000 ng/mL (r>0.997 for all drugs). Accuracy was assessed by correlation of the calibrators with proficiency testing samples spiked with known drug concentrations and yielded results within 8% of the target values.

Standards of zalcitabine (ddC), didanosine (ddI) and zidovudine (AZT) were purchased from Sigma (St. Louis, Mo., USA). Primary standards of efavirenz, indinavir, nelfinavir, nevirapie, ritonavir, saquinavir, lamivudine, abacavir and stavudine were obtained from the NIH Aids Reagent Reference Program (McKessonHBOC BioServices, Rockville, Md.) while standards for amprenavir, delavirdine, lopinavir and tenofovir were isolated from commercially available tablets/capsules and characterized by H-NMR, UV spectroscopy and elemental analysis. Methanol, acetonitrile, and ammonium acetate were purchased from Sigma and were of HPLC grade.

Stock solutions were prepared separately to obtain a concentration of 1.0 mg mL⁻¹ of each drug (total of 16 drugs) with methanol as a solvent. The working standard solution was prepared by mixing equal amounts of each drug and diluting with serum to obtain a solution containing 10,000 ng mL⁻¹ of each drug. The calibration curve consisted of a blank and five standards (10,000 ng ml⁻¹, 5,000 ng ml⁻¹, 2500 ng mL⁻¹, 1000 ng mL⁻¹, 100 ng mL⁻¹). Serum or plasma samples were obtained from hospital patients and proficiency testing samples (PT) from the International Quality Control Program for Therapeutic Drug Monitoring in HIV Infection (University Medical Center Nijmegan, Department of Clinical Pharmacy, PO Box 9191, 6500 HB Nijmegan, The Netherlands). Table 8 shows the concentration range covered by the six PT samples. A solution of 0.15 mg L⁻¹ of cimetidine dissolved in acetonitrile was used as the stock internal standard for both negative and positive MS/MS modes. The stock solution was diluted 2500 fold with acetonitrile to give the working internal standard solution.

For sample preparation, 80 μL of serum or heparinized plasma were placed into a 1.5 mL conical plastic Eppendorf test tubes containing 120 μL of internal standard solution. The tubes were capped, vortexed vigorously for 30 s and centrifuged at 14,000 g for 10 min. The supernatant was transferred into autosampler vials for injection into the LC/MS/MS system. Sample preparation was performed at room temperature. Plasma obtained from HIV-infected patients was heated for 30 min at 56° C. to deactivate the HIV virus [26].

This demonstrates a simple, expeditious and automated method of preparing for analysis a complex matrix containing a plurality of antiretroviral drugs.

Ionization and Spectrometry

An API-2000™ tandem mass spectrometer (SCIEX, Toronto, Canada) equipped with atmospheric pressure chemical ionization (APCI, heated nebulizer) source, two Perkin-Elmer PE-₂₀₀™ series micropumps and autosampler (Perkin-Elmer, Norwalk, Conn., USA) was used to perform the analysis. An injection volume of 20 μL was used for all samples. Data processing was performed on Analyst 1.1 software package (SCIEX). The main working parameters of the mass spectrometer and detailed procedure have been described in Example 1, above.

Quantification of tenofovir by multiple reaction monitoring yielded an optimal transition in the positive ionization mode of 520.3/270.0. See Table 3 for the transitions for the other drugs.

Calibration curves for all 16 anti-HIV drugs showed good linearity between a concentration range of 100-10,000 ng mL⁻¹ (r>0.997 for all drugs). The eight antiretrovirals present in the proficiency testing samples and their concentrations from low to high are listed in Table 8. Correlation of calibrators with the proficiency testing samples yielded the regression parameters listed in Table 7.

A method for obtaining extensive correlations for 9 of the 16 drugs with another method are described above in Example 1 and elsewhere [25]. At Children's National Medical Center in Washington, D.C., the therapeutic range for PIs is 150 ng mL⁻¹-6000 ng mL⁻¹, except for lopinavir which should be maintained at concentrations between 150-12,000 ng mL⁻¹ [27]. For the NNRTIs, the currently recommended therapeutic range is between 1200 and 7000 ng mL⁻¹ [27].

This demonstrates a simple, automated and expeditious method of simultaneously analyzing a plurality of antiretroviral drugs by mass spectrometry. Good linearity was obtained for all 16 drugs (r>0.997). The assay was further assessed by correlating the calibrators with 6 serum samples obtained from a proficiency testing program that included eight of the sixteen drugs measured in the procedure. The targets were listed from low to high for each drug (Table 8). Good linearity (r>0.944 for all drugs) and accuracy (within 8% of target value of proficiency testing samples) was observed for all drugs tested. Drug comparison studies for the other eight drugs was not performed as it was not possible to obtain samples for calibration from other laboratories despite many and continuing efforts to do so. The precision obtained with this revised procedure was the same as that of example 1.

### Example 3

### Serum Standard Matrix

The following example is provided to illustrate how the method can be applied to new antiretrovirals, such as FIs. Standard solution and sample preparation and analysis is performed in accordance with the method set out in Example 2. A standard solution of a FI, for example T-20, is also prepared, with MRM transitions for T-20 established by a method known to one skilled in the art.

### Example 4

### Therapeutic Drug Monitoring (TDM) for Patients with HIV Infection and Correlation between Measurements of Free Plasma Drug Concentration and Saliva Drug Concentration

Excellent reviews of therapeutic drug monitoring in HIV infection have been published (7, 12, 27 and 35). Much data exists today to show that the PIs and NNRTIs meet many of the conventional requirements for TDM. These include a good correlation between serum concentration and pharmacological effect as measured by CD4 counts and viral loads, a concentration above which toxicity is commonly manifested and a reliable method of drug quantitation (36-39). Tables 9 and 10 show a summary of some pharmacokinetic parameters of antiretroviral drugs and a comparison of HPLC and tandem mass spectrometry methods for antiretroviral drug measurements. One of the main messages supported by data in Table 9 is that the ideal sampling time is the steady state trough concentration which is the drug concentration just before the next drug dose after at least 5 half lives have elapsed and that for most of the antiretrovirals this would be at least 2 days after initiation of the drug therapy. Exceptions to this rule have to be made for nevirapine (an NNRTI) and saquinavir (a PI), both of which have longer half-lives and should not be monitored until 4 days after initiation of the drug regimen unless there are signs and symptoms of drug toxicity. One of the main reasons for TDM of antiretrovirals is the well-known lack of compliance of patients on long term therapy for chronic illnesses. For HIV/AIDS patients this lack of adherence to their drug regimens commonly occurs in 33-60% of patients (12, 27, 35). When failure of antiretroviral therapy occurs, assessment of compliance (adherence) by TDM, prior to resistance testing can be very important. Documentation of ongoing antiretroviral drug presence make the results of both genotypic or phenotypic HIV resistance tests more meaningful. Drug-drug interactions and interpatient variability in drug handling are other major reasons for TDM in this population. Table 10 shows a comparison between HPLC and tandem mass spectrometry methods for antiretroviral drug measurement. It is clear that the latter provides for a simultaneous measurement of 16 drugs in a short time frame with minimal sample preparation (15, 40), is reliable, more sensitive and also more specific than HPLC methods (16, 41 ).

Tentative therapeutic ranges: The minimal effective concentrations (MECs) for the PIs vary between 60 and 100 ng/mL. At Children's National Medical Center the lower limit of the therapeutic range for the PIs is taken as 150 ng/mL, which is based on literature PK values and the locally observed mean trough levels for current pediatric dosing of PIs, as recommended by the Public Health Service The recommended upper limit concentration is less than 6,000 ng/mL, except for lopinavir, which should be maintained at concentrations <12,000 ng/mL. For the NNRTIs, the recommended tentative ranges are between 1,200-7,000 ng/mL.

Proficiency testing (PT): Available through the International Quality Control Program for Therapeutic Drug Monitoring in HIV Infection (University Medical Center Nijmegan, Department of Clinical Pharmacy, PO Box 9101, 6500 HB Nijmegan, The Netherlands). This program provides PT Testing for some 8/9 drugs (PIs and NNRTIs) twice annually. The College of American Pathologists will probably have a broader program covering 16 antiretrovirals twice a year and available in 2005.

Protein binding and free drug measurement: An Institutional Review Board (IRB) approved protocol was developed for the assessment of free drug concentrations by measuring both the saliva and free drug in plasma utilizing the tandem mass spectrometric methods for plasma or serum measurement. The methods work equally well on either plasma or serum. A bigger sample is injected for the free drug measurement (50 uL vs 10 uL for plasma/serum.). Free drug concentrations can be measured by using either equilibrium dialysis or, more commonly, devices with molecular cutoff filters such as the Amicon Centrifree micropartition system (Millipore Corporation, Bedford, Mass.) or the Worthington Diagnostics “ultrafree” system (Worthington, Jacksonville, Fla.). Plasma ultrafiltrate is obtained by centrifuging 1 mL of plasma in Amicon system and then deproteinising as described for serum/plasma previously (15, 40). Tables 11-13 show the correlations between free plasma drug and saliva drug with total plasma drug concentrations for some of the PIs, NRTIs and NNRTIs. This data confirms previous reports that the PIs are strongly protein bound (7, 12, 27 and 35). The correlations obtained for the free drug or saliva drug concentration with the total drug concentrations are generally very good. For the PIs (Table 11) good correlations were found for lopinavir and ritonavir but not for nelfinavir. For the NRTIs (Table 12) good correlations were found for didanosine, zidovudine and stavudine. Finally for the NNRTIs (Table 13) good correlations were found for nevirapine. This suggest that salivary monitoring is possible in place of plasma monitoring to assess patient compliance with the drug regimen and also optimization of patient outcomes employing salivary TDM.

Conclusions: It is estimated that there are 40 million people with epilepsy worldwide and 750,000 transplant recipients. In contrast there are 42 million people worldwide infected with HIV-1. Of these, currently about 1 million are receiving antiretroviral therapy. TDM is well accepted for both the optimization of patient outcomes in patients with epilepsy and in post-transplant patients but has been underutilized for patients with HIV infection, although significant progress has been made in recent years. This example has overviewed the field of TDM in HIV infection and provided current data on salivary/free drug monitoring which has important potential as a non-invasive means of assessing compliance and of optimizing therapy for patients in this fast developing area of TDM with HIV infection.

While the above detailed description describes the exemplifying embodiments of the present invention, it should be understood that the present invention is susceptible to modifications, variations and alterations without deviating from the scope of the invention.

TABLE 1
Marketed anti-HIV drugs

| | | Non- | | |
|---|---|---|---|---|
| | | Nucleoside | Nucleotide | |
| | Nucleoside | Reverse | Reverse | Fusion/ |
| Protease | Reverse | Transcriptase | Trans- | Entry |
| Inhibitors | Transcriptase | Inhibitors | criptase | Inhibitors |
| (PI) | Inhibitors (NRTI) | (NNRTI) | (NtRTI) | (FI/EI) |
| Amprenavir | Abacavir (ABC) | Delavirdine | Tenofovir | T-20 |
| Indinavir | Didanosine (ddl) | Efavirenz | | Peptide T |
| Nelfinavir | Lamivudine (3TC) | Nevirapine | | |
| Ritonavir | Stavudine (d4T) | | | |
| Saquinavir | Zalcitabine (ddC) | | | |
| Lopinavir | Zidovudine (AZT) | | | |
| Atazanavir | | | | |
| (Reyataz ™) | | | | |

TABLE 2
Estimated limit of quantitation (LOQ) versus known
trough concentrations of anti-HIV drugs

| | | Trough |
|---|---|---|
| | Estimated LOQ | Concentration |
| Drug | (ng/mL) | (ng/mL) |
| Efavirenz | 1 | — |
| Zidovudine (AZT) | 3 | <20 |
| Stavudine (d4T) | 3 | 20 |
| Indinavir | <1 | 100 |
| Abacavir | 1 | — |
| Nelfinavir | <1 | 1000 |
| Delavirdine | <1 | 3000-8000 |
| Saquinavir | <1 | 15-40 |
| Nevirapine | 1 | 3000 |
| Lamivudine (3TC) | 1 | 100-1000 |
| Ritonavir | <1 | 1000 |
| Amprenavir | <1 | — |
| Zalcitabine (ddC) | 1 | Undetectable |
| Didanosine (ddl) | 1 | 100-300 |
| Lopinavir | <1 | — |

TABLE 3
MS/MS ion transitions and modes of analysis

| Drug | Ionization mode | MS/MS transition |
|---|---|---|
| Efavirenz | Negative | 314.2/69.0 |
| Zidovudine (AZT) | Negative | 266.2/222.8 |
| Stavudine (d4T) | Negative | 223.0/42.0 |
| Indinavir | Positive | 614.5/421.3 |
| Abacavir | Positive | 287.1/191.1 |
| Nelfinavir | Positive | 568.3/330.1 |
| Delavirdine | Positive | 457.2/221.1 |
| Saquinavir | Positive | 671.4/570.3 |
| Nevirapine | Positive | 267.2/226.1 |
| Lamivudine (3TC) | Positive | 230.1/112.0 |
| Ritonavir | Positive | 721.3/268.2 |
| Amprenavir | Positive | 506.3/245.2 |
| Zalcitabine (ddC) | Positive | 212.1/112.0 |
| Didanosine (ddl) | Positive | 237.1/137.1 |
| Lopinavir | Positive | 629.3/447.3 |
| Tanofovir | Positive | 520.3/270.0 |

TABLE 4
Tandem mass spectrometer main working parameters

| Parameter | Value |
|---|---|
| Nebulizer temperature, ° C. | 480 |
| Dwell time per transition, msec | 80 |
| Nebulizer gas (Gas 10), psi | 85 |
| Auxiliary gas (Gas 2), psi | 20 |
| Curtain gas, psi | 30 |
| Nebulizer current, μA | 2 |
| Ion energy, V | 0.8 |

TABLE 5
Inter-day accuracy and precision data

| | | Mean(%) | | | % CV | | |
|---|---|---|---|---|---|---|---|
| | | 10 | 100 | 500 | 10 | 100 | 500 |
| | n | ng/ml | ng/ml | ng/ml | ng/ml | ng/ml | ng/ml |
| Efavirenz | 20 | 104.5 | 102.6 | 101.4 | 9.9 | 8.7 | 6.2 |
| Zidovudine (AZT) | 20 | 95.0 | 96.1 | 103.2 | 9.5 | 9.3 | 5.1 |
| Stavudine (d4T) | 20 | 104.7 | 101.5 | 102.6 | 8.9 | 7.9 | 8.0 |
| Indinavir | 20 | 104.1 | 98.2 | 101.0 | 7.6 | 6.0 | 3.2 |
| Abacavir | 20 | 96.8 | 97.9 | 97.5 | 8.7 | 6.2 | 5.5 |
| Nelfinavir | 20 | 95.9 | 104.3 | 102.0 | 8.4 | 6.3 | 4.2 |
| Delavirdine | 20 | 104.3 | 96.9 | 99.8 | 8.2 | 5.2 | 6.0 |
| Saquinavir | 20 | 103.9 | 101.8 | 98.7 | 9.5 | 7.1 | 4.1 |
| Nevirapine | 20 | 95.5 | 103.1 | 101.9 | 6.9 | 4.2 | 5.9 |
| Lamivudine (3TC) | 20 | 98.7 | 103.0 | 100.8 | 8.8 | 4.9 | 2.1 |
| Ritonavir | 20 | 104.1 | 98.2 | 97.5 | 7.6 | 6.3 | 3.9 |
| Amprenavir | 20 | 103.5 | 99.4 | 99.0 | 8.6 | 7.4 | 4.5 |
| Zalcitabine (ddC) | 20 | 96.4 | 101.9 | 103.0 | 9.8 | 7.1 | 3.9 |
| Didanosine (ddI) | 20 | 97.7 | 103.6 | 100.1 | 9.7 | 6.1 | 6.5 |
| Lopinavir | 20 | 95.0 | 103.9 | 102.9 | 5.2 | 1.8 | 1.8 |

TABLE 6
Linear regression parameters calculated from method comparisons

| | | | | X-in- | | |
|---|---|---|---|---|---|---|
| | n | Slope | Y-intercept | tercept | r | S_{y,x} |
| Amprenavir | 16 | 0.54 ± 0.06 | 270.8 ± 146.1 | −498.7 | 0.911 | 304.0 |
| Delavirdine | 10 | 0.79 ± 0.03 | 266.6 ± 231.4 | −338.1 | 0.993 | 425.9 |
| Nevirapine | 28 | 0.78 ± 0.07 | 506.5 ± 230.3 | −546.0 | 0.928 | 428.5 |
| Lopinavir | 32 | 0.87 ± 0.05 | 558.0 ± 381.1 | −642.9 | 0.949 | 944.2 |
| Efavirenz | 15 | 1.15 ± 0.08 | 150.8 ± 188.8 | −131.5 | 0.971 | 398.4 |
| Ritonavir | 52 | 0.96 ± 0.02 | −4.8 ± 44.4 | −5.05 | 0.992 | 266.7 |
| Saquinavir | 26 | 0.91 ± 0.03 | 11.0 ± 18.4 | −12.0 | 0.989 | 71.08 |
| Nelfinavir | 33 | 0.73 ± 0.02 | 88.5 ± 41.5 | −121.7 | 0.988 | 146.8 |
| Indinavir | 98 | 1.16 ± 0.02 | −77.1 ± 34.4 | −66.6 | 0.983 | 252.2 |

S_{y,x} - standard deviation of the residuals.

TABLE 7
Linear regression parameters calculated from comparison
of our results with the 5 or 6 known target
concentrations in proficiency testing samples

| Drug | n | Slope | Y-intercept | r | S_{y,x} |
|---|---|---|---|---|---|
| Amprenavir | 6 | 0.93 ± 0.02 | −14.0 ± 106.6 | 0.998 | 180.5 |
| Indinavir | 5 | 0.92 ± 0.06 | −30.3 ± 436.1 | 0.993 | 699.7 |
| Lopinavir | 6 | 1.03 ± 0.05 | 268.6 ± 340.9 | 0.995 | 490.0 |
| Nelfinavir | 6 | 1.07 ± 0.02 | −161.9 ± 44.48 | 1.000 | 77.06 |
| Ritonavir | 6 | 0.94 ± 0.03 | −77.5 ± 205.2 | 0.998 | 363.0 |
| Saquinavir | 5 | 0.95 ± 0.03 | −50.93 ± 91.29 | 0.998 | 141.4 |
| Nevirapine | 5 | 0.94 ± 0.19 | −67.3.1 ± 727.9 | 0.944 | 958.4 |
| Efavirenz | 5 | 1.09 ± 0.02 | 138.7 ± 109.2 | 0.999 | 144.4 |

TABLE 8
Target concentrations of proficiency testing (PT) samples

| | Concentration Level (ng mL⁻¹) | | | | | |
|---|---|---|---|---|---|---|
| Drug | Low | | Medium | | High | |
| PT target values | (1) | (2) | (3) | (4) | (5) | (6) |
| Amprenavir | 220 | 240 | 2150 | 2160 | 8600 | 7200 |
| Indinavir | 170 | 130 | 2330 | | 10100 | 11720 |
| Lopinavir | 1000 | 1170 | 5000 | 4670 | 10000 | 11680 |
| Nelfinavir | 230 | 320 | 1640 | 1630 | 2120 | 6360 |
| Ritonavir | 270 | 240 | 2670 | 2420 | 13400 | 9700 |
| Saquinavir | 120 | 110 | 1410 | | 3400 | 5080 |
| Nevirapine | 500 | 1090 | 3900 | 3160 | 7800 | |
| Efavirenz | 460 | | 1000 | 3700 | 7300 | 6590 |

TABLE 9
PHARMACOKINETIC PARAMETERS FOR ANTIRETROVIRALS

| | C max. | C min. | | Protein | |
|---|---|---|---|---|---|
| Drug | mg/L | mg/L | t ½ (h) | Bindings % | Vd L/Kg |
| NRTIs | | | | | |
| Didanosine | 24 | 0.1 | 1.5 | <5 | 0.8 |
| Lamivudine | 1.8 | 0.1 | 6 | <5 | NA |
| Stavudine | 2.0 | 0.02 | 1.1 | <5 | 1.0 |
| Zalcitabine | <0.025 | undetectable | 2.0 | <5 | 0.5 |
| Zidovudine | 1.8 | <0.02 | 1.2 | <5 | 1.4 |
| NNRTIs | | | | | |
| Delavirdine | 16 | 3.0 | 6 | 99 | NA |
| Nevirapine | 20 | 3.0 | 30 | 60 | 1.2 |
| PIs | | | | | |
| Indinavir | 10 | 0.1 | 1.8 | NA | NA |
| Nelfinavir | 4 | 1.0 | 5 | 98 | 4 |
| Ritonavir | 14 | 1.0 | 4 | 98 | 0.4 |
| Staquinavir | 0.5 | 015 | 12 | 98 | 10 |

TABLE 10
HPLC vs. Tandem-MS

| Parameters | HPLC | Tandem-MS |
|---|---|---|
| Sample volume | 0.5-1.0 mL | 0.08 mL |
| Sensitivity | 10-100 ug/L | 1 ug/L |
| Sample prep | Lengthy | Just precipitation of |
| | | proteins |
| Chromatography time | 15 min-1 hr | <5 min. |
| General | Different methods | Universal method |
| | needed for different | |
| | drugs | |

TABLE 11
Pls Saliva(s) or Free Plasma(f) vs Total Plasma(tp)

| DRUG | s or f vs tp | n | r |
|---|---|---|---|
| lopinavir | s = 0.05tp + 194 | 11 | 0.777 |
| | f = 0.019tp − 59 | 6 | 0.993 |
| ritonavir | s = 0.043tp + 109 | 5 | 0.991 |
| nelfinavir | s = 0.0026tp + 78 | 7 | 0.047 |

TABLE 12
NRTIs Saliva(s) or Free Plasma(f) vs Total Plasma (tp)

| DRUG | s or f vs tp | n | r |
|---|---|---|---|
| didanosine | s = 0.29tp + 20 | 6 | 0.912 |
| | f = 0.19tp + 47 | 6 | 0.749 |
| zidovudine | f = 0.14tp + 27 | 8 | 0.907 |
| stavudine | s = 093tp + 38 | 17 | 0.909 |
| | f = 0.66tp − 4 | 17 | 0.924 |

TABLE 13
NNRTIs Saliva(s) or Free Plasma (f) vs Total Plasma(tp)

| DRUG | s or f vs tp | n | r |
|---|---|---|---|
| nevirapine | s = 063tp + 0.912 | 10 | 0.901 |
| | f = 0.36tp + 483 | 14 | 0.909 |

Cited References
1 Volberding P A, Deeks S G. Antiretroviral therapy for HIV infection: promises, and problems. JAMA 1998;279:1343-4.
2 Temesgen Z, Wright A J. Recent advances in the management of human immunodeficiency virus infection. Mayo Clinic Proc 1997;72: 854-9.
3 Ledergerber B, Egger M, Opravil M. et al. Clinical progression and virological failure on highly active antiretroviral therapy in HIV-1 patients: a prospective cohort study. Lancet 1999;353:863-8.
4 Flexner C. Dual protease inhibitor therapy in HIV-infected patients: pharmacological rationale, and clinical benefits. Annu Rev Pharmacol Toxicol 2000;40:649-74.
5 Dresser G K, Spence J D, Bailey D G. Pharmacokinetic-pharmacodynamic consequences, and clinical relevance of cytochrome P450 3A inhibition. Clin Pharmacokinet 2000;38:41-57.
6 Piscitelli S C, Gallicano K D. Interactions among drugs for HIV, and opportunistic infections. N Engl J Med 2001;344:984-96.
7 Back D J, Khoo S H, Gibbons S E, Barry M G, Merry C. Therapeutic drug monitoring of antiretrovirals in human immunodeficiency virus infection. Ther Drug Monit 2000;22:122-6.
8 Barry M G, Merry C, Lloyd J, Halifax K, Carey P, Mulcahy F, Back D J. Variability in trough plasma saquinavir concentrations in HIV patients. A case for therapeutic drug monitoring. Br J Clin Pharmacol 1998;45:501-2.
9 Kastrissios H, Suarez J R, Hammer S, Katzenstein D, Blaschke T F. The extent of non-adherence in a large AIDS clinical trial using plasma dideozynucleoside concentrations as a marker. AIDS (London) 1998;12:2305-11.
10 Paterson D L, Swindells S, Mohr J, Brester M, Vergis E N, Squier C, Wagener M M, Singh N. Adherence to protease inhibitor therapy, and outcomes in patients with HIV infection. Ann Intern Med 2000;133: 21-30.
11 Falloon J, Piscitelli S, Vogel S, Sadler B, Mitsuya H, Kavlick M F, Yoshimura K, Rogers M, LaFon S, Manion D J, Lane H C, Masur H. Combination therapy with amprenavir, abacavir, and efavirenz in human immunodeficiency virus (HIV)-infected patients failing a protease-inhibitor regimen: pharniacokinetic drug interactions, and antiviral activity. Clin Infect Dis 2000;30:313-8.
12 Van Heeswijk R P G. Critical issues in therapeutic drug monitoring of antiretroviral drugs. Ther Drug Monit 2002;24:323-31.
13 College of American Pathologists, Proficiency Testing Data.
14 Ghoshal A, Soldin S. Improved Method for Concurrent Quantification of Antiretrovirals by Liquid Chromatography-Tandem Mass Spectrometry, Unpublished.
15 Volosov A, Alexander C, Ting L, Soldin S J. Simple rapid method for quantification of antiretrovials by liquid chromatography—tandem mass-spectrometry. Clin Biochem 2002; 35:99-103.
16 Moyer T P, Temesgen Z, Enger R, Estes L, Charlson J. Oliver L, Wright A. Drug monitoring of antiretroviral therapy for HIV-I infection: method validation, and results of a pilot study. Clin Chem 1999;45:1465-76.
17 Villani P, Feroggio M, Gianelli L, Bartolli A, Montagna M, Maserati R, Regazzi M B. Antiretrovirals: Simultaneous Determination of Five Protease Inhibitors and Three Nonnucleoside Transcriptase Inhibitors in Human Plasma by a Rapid High-Performance Liquid Chromatography-Mass Spectrometry Assay, Ther Drug Monit 2001;23(4):380-388.
18 Boffito M, Hoggard P, Reynolds H, Bonara S, Meaden E R, Sinicco A, Di Perri G, Back D J. The Unbound Percentage of Saquinavir and Indinavir Remains Constant Throughout the Dosing Interval in HIV Positive Subjects, J Clin Pharmacol 2002;54:262-268.
19 Bouley M, Briere C, Padoin C, Petitjean O, Tod M. Sensitive, and rapid method for the simultaneous quantification of the HIV-protease inhibitors indinavir, nelfinavir, ritonavir, and saquinavir in human plasma by reversed-phase liquid chromatography. Ther Drug Monit 2001;23:56-60.
20 Yamada H, Kotaki H, Nakamura T, Iwamoto A. Simultaneous determination of the HIV protease inhibitors indinavir, amprenavir, saquinavir, ritonavir, and nelfinavir in human plasma by high-performance liquid chromatography. J Chromatogr B 2001;755:85-9.
21 Agrofoglio L A, Cahours X, Tran T T, Dessans H, Kieda C, Morin P. Analysis of anti-HIV nucleoside inhibitors by capillary electrophoresis-electrospray ionization mass spectromerty. Nucleosides Nucleotides Nucleic Acids 2001;20:375-81.
22 Poirier J M, Radembino N, Robidou P, Jaillon P. Simultaneous determination of the five HIV-protease inhibitors: amprenavir, indinavir, nelfinavir, ritonavir, and saquinavir in human plasma by solid-phase extraction, and column liquid chromatography. Ther Drug Monit 2000;22:465-73.
23 Chi J, Jayewardene A, Stone J A, Motoya T, Aweeka F, Simulataneous Determination of Five HIV Protease Inhibitors, Nelfinavir, Indinavir, Ritonavir, Saquinavir, and Amprenavir in Human Plasma by LC/MS/MS. J Pharm Biomed Anal 2002;30: 675-684.
24 Gao W, Kishida T, Kimura K, Kageyama M, Sumi M, Yoshikawa Y, Shibata N, Takida K. Sensitive and Simultaneous Determination of HIV Protease Inhibitors in Rat Biological Samples by Liquid Chrornatography-Mass Spectrometry. Biomed Chromatogr 2002;16: 267-273.
25 Volosov A, Napoli K L, Soldin S J. Simultaneous simple, and fast quantification of three major immunosuppressants by liquid chromatography-tandem mass-spectrometry. Clin Biochem 2001;34:285-90.
26 Dasgupta A, Wells A, Chow L. Effect of heating human seraat a temperature necessary to deactivate human immunodeficiency virus on measurement of free phenytoin, free valproic acid, and free carbamazepine concentrations. Ther Drug Monit 1999;21:421-5.
27 Soldin O P, Elin R J, Soldin S J. Therapeutic drug monitoring in human immunodeficiency virus/acquired immunodeficiency syndrome. Quo vadis? Ach pathol Lab Med 2003; 127: 102-104.
28 Ghoshal A, Soldin S. Imx Tacrolimus II Assay: Is it Reliable at Low Blood Concentrations? A Comparison with Tandem MS/MS, Clin. Biochem. 2002;389-392.
29 De Clercq E. New developments in anti-HIV chemotherapy. Biochim Biophys Acta 2002;1 587:258-275.
30 Gerber J G, Acosta E P. The potential role of therapeutic drug monitoring in the treatment of HIV infection. Topics HIV Med 2002; 10: 27-32.
31 Hallworth M, Capps N. Therapeutic drug monitoring and clinical biochemistry. Washington D.C., MCC Press, 1994.
32 Schumacher G E. Therapeutic drug monitoring. Stanford, Conn., Appleton & Lange, 1995.
33 Wong S H Y, Sunshine I. Eds: Handbook of analytical therapeutic drug monitoring and toxicology. Washington, D.C., MCC Press, 1995.
34 Israili Z H, Dayton P G. Human alpha-1-glycoprotein and its interactions with drugs. Drug Metab Rev 2001; 33:161-235.
35. Dasgupta A, Okhuysen P C. Pharmacokinetic and other drug interactions in patients with AIDS. Ther Drug Monit. 2001:23;591-605.
36. Gerber J G. Using pharmacokinetics to optimize antiretroviral drug-drug interactions in the treatment of human immunodeficiency virus infection. Clin Infect Dis.2000;30(suppl 2):S123-S129.
37. van Heeswick R P G, Veldkamp A I, Mulder J W, et al. The steady-state pharmacokinetics of nevirapine during once daily and twice daily dosing in HIV-1 infected individuals. AIDS 2000:14;F77-F82.
38. Durant J, Clevenbergh P, Garrafo R, et al. Importance of protease inhibitor plasma levels in HIV-infected patients treated with genotypic-guided therapy: pharmacological data from the Viradept study. AIDS.2000;14:1333-1339.
40. Burger D, Hugen P, Droste J et al. Therapeutic drug monitoring (TDM ) of nelfinavir (NFV) and indinavir (IDV) in treatment—naïve patients improves therapeutic outcome after 1 year: results from ATHENA. In: Abstracts of the First IAS Conference on HIV Pathogenesis and Treatment; 2001; Buenos Aires, Argentina. Abstract 30.
40. Ghoshal A K, Soldin S J. Improved Method for Concurrent Quantification of Antiretrovirals by Liquid Chromatography-Tandem Mass Spectrometry. Ther Drug Monit. 2003:25:541-3.
41. Keil K, Frerichs V A, DiFrancesco R, Morse G. Reverse Phase High-Performance Liquid Chromatography Method for the Analysis of Amprenavir, Efavirenz, Indinavir, Lopinavir, Nelfinavir and its Active Metabolite (M8), Ritonavir, and Saquinavir in Heparinized Human Plasma.Ther Drug Monit. 2003;25:340-6.

1. A method for mass spectrometric analysis of a sample comprising at least two antiretroviral drugs from at least two classes of antiretroviral drugs, the method comprising the steps:
(a) providing a sample comprising at least two antiretroviral drugs from at least two classes of antiretroviral drugs;
(b) introducing the sample comprising at least two antiretroviral drugs from at least two classes of antiretroviral drugs into a mass spectrometer; and
(c) analyzing the sample using the mass spectrometer.

2. The method according to claim 1 wherein the mass spectrometer is a tandem-mass spectrometer.

3. The method of claim 1 further comprising a step of deproteinating the sample.

4. The method according to claim 1 wherein the classes of antiretroviral drugs are selected from the group consisting of PIs, NRTIs, NtRTIs, NNRTIs and FIs.

5. The method according to claim 1 wherein the antiretroviral drugs are selected from the group consisting of amprenavir, indinavir, nelfinavir, ritonavir, saquinavir, lopinavir, abacavir, didanosine, lamivudine, stavudine, zalcitabine, zidovudine, delavirdine, efavirenz, nevirapine, tanofavir, atazanavir, peptide T and T-20.

6. The method according to claim 1 wherein the sample containing at least two antiretroviral drugs is obtained from a biological matrix selected from the group consisting of plasma, serum, urine and saliva.

7. The method of claim 6 wherein the biological matrix is plasma.

8. The method of claim 6 wherein the biological matrix is serum.

9. The method of claim 6 wherein the biological matrix is saliva.

10. The method according to claim 1 wherein size of the sample containing at least two antiretroviral drugs is about 80 μL.

11. The method according to claim 3 wherein the step of deproteinating the sample comprises:
(a) adding acetonitrile to the sample;
(b) vortexing the sample; and
(c) subjecting the sample to centrifugation.

12. The method according to claim 3 wherein the step of deproteinating the sample comprises subjecting the sample to precipitation with an agent selected from the group consisting of methanol, ethanol and salt.

13. The method of claim 1 further comprising a step of cleaning the sample.

14. The method according to claim 13 wherein the step of cleaning the sample comprises introducing the sample to a chromatography apparatus and eluting the sample.

15. The method according to claim 2 wherein the tandem-mass spectrometer is selected from the group consisting of API 2000™, API 3000™ and API 4000™.

16. The method according to claim 1 wherein the step of analyzing the sample using a mass spectrometer comprises a step of atmospheric pressure chemical ionization using a heated nebulizer.

17. The method according to claim 1 wherein the step of analyzing the sample using a mass spectrometer comprises multiple reaction monitoring.

18. The method according to claim 1 which does not include chromatographic separation of the antiretroviral drugs.

19. The method according to claim 1 wherein the sample comprises a plurality of antiretroviral drugs and they are analyzed simultaneously.

20. The method according to claim 1 wherein the sample comprises a plurality of antiretroviral drugs and they are analyzed sequentially.

21. A method for therapeutic drug monitoring in patients with HIV infection, comprising:
(a) providing a sample comprising at least two antiretroviral drugs from at least two classes of antiretroviral drugs;
(b) introducing the sample comprising at least two antiretroviral drugs from at least two classes of antiretroviral drugs into a mass spectrometer; and
(c) analyzing the sample using the mass spectrometer.

22. The method according to claim 21 wherein the mass spectrometer is a tandem-mass spectrometer.

23. The method of claim 21 further comprising a step of deproteinating the sample.

24. The method according to claim 21 wherein the classes of antiretroviral drug is selected from the group consisting of PIs, NRTIs, NtRTIs, NNRTIs and FIs.

25. The method according to claim 21 wherein the antiretroviral drug is selected from the group consisting of amprenavir, indinavir, nelfinavir, ritonavir, saquinavir, lopinavir, abacavir, didanosine, lamivudine, stavudine, zalcitabine, zidovudine, delavirdine, efavirenz, nevirapine, tanofavir, atazanavir, peptide T and T-20.

26. The method according to claim 21 wherein the sample containing at least two antiretroviral drugs is obtained from a biological matrix selected from the group consisting of plasma, serum, urine and saliva.

27. The method of claim 21 further comprising a step of cleaning the sample.

28. A system for the mass spectrometric analysis of a sample comprising at least two antiretroviral drugs from at least two classes of antiretroviral drugs, comprising:
(a) reagents for deproteinating the sample;
(b) reagents for analyzing the sample by mass spectrometry; and
(c) a mass spectrometer.

29. A kit for use in mass spectrometric analysis of a sample comprising at least two antiretroviral drugs from at least two classes of antiretroviral drugs, comprising:
(a) reagents for deproteinating the sample;
(b) reagents for analyzing the sample by mass spectrometry;
(c) instructions for analyzing the sample using a mass spectrometer.

30. The kit according to claim 29 further comprising:
(a) mobile phase solutions;
(b) a chromatography column; and
(c) a quality control specimen.

31. Use of a mass spectrometer for sequentially or simultaneously analyzing a sample containing at least two antiretroviral drugs from at least two classes of antiretroviral drugs.

32. The use of claim 31 wherein the mass spectrometer is a tandem mass spectrometer.
